(19) United States
(12) Patent Application Publication
(10) Pub. No.: US 2009/0227682 A1
(43) Pub. Date: Sep. 10, 2009

(54) XETINE COMPOSITIONS

(75) Inventors: Benoit Schmitt, Irvine, CA (US); Laurent Imler, Irvine, CA (US)

Correspondence Address: HARNESS, DICKEY & PIERCE, P.L.C., P.O. BOX 828, BLOOMFIELD HILLS, MI, 48303, US

(73) Assignee: Pharma PASS II LLC, Irvine, CA (US)

(21) Appl. No.: 12/041,995

(22) Filed: Mar. 4, 2008

(51) Int. Cl. A61K 31/137; C07C 217/62; A61P 25/24

(52) U.S. Cl. 514/651; 564/443

(57) ABSTRACT

The invention provides a xetine or salt thereof composition having a dissolution in a phosphate buffer at pH 6.8, using USP basket 10 Mesh at 75 rpm, of at least 15% at 60 minutes, of at least 40% at 120 minutes, of at least 70% at 300 minutes.

### FIELD OF THE INVENTION

The invention relates to an improved xetine compositions and especially to an atomoxetine improved composition.

### BACKGROUND OF THE INVENTION

Xetines are currently among therapeutically effective drugs available for ameliorating depression. The mechanism is explained in Neuropharmacology mechanism of depression, Diseases of the Nervous System, 841-46 (1977) and in Review of the pharmacology of Existing Antidepressant, Br J. Clin. Pharmac. 4, 575-685 (1977). Such condition occurs when the nervous system becomes unable to pass impulses between neurons in adequate numbers at adequate speeds. One of the major causes of depression has been found to be a condition wherein inadequate monoamines are present in the central nervous system. The most important of these amines are norpinephrine, serotonin and dopamine. Xetines are part of the class of drugs called the monoamines reuptake inhibitors that have been found to be effective in ameliorating this condition, by inhibiting the reuptake of the amines by the synapses, the nerve terminals from which the amines were released. In addition, since norepinephrine is believed to play a role in Attention-Deficit-Hyperactivity Disorders (ADHD), some of the xetines have thus found second applications for these conditions.

Specific examples of xetines include: paroxetine, tomoxetine, atomoxetine, reboxetine, dapoxetine, fluoxetine, sluoxetine and duloxetine. The mentioned xetines have recognized or suspected reuptake inhibiting activities for one or more of the neropinephrine, serotonine, or dopamine monoamines.

Preferred, atomoxetine (ATM) including its pharmaceutical salts is disclosed in EP. Pat. No. 052 492 owned by Eli Lilly and Company. Atomoxetine is the (R)-(−) enantiomer of tomoxetin and the common name of the chemical compound (3R)-N-methyl-3-(2-methylphenoxy)-3-phenyl-propan-1-amine. It is used for the treatment of ADHD as disclosed in EP patent No 721 777 and acts as selective norepinephrine inhibitor, which is believed to exert its clinical effects primarily via selective inhibition of the presynaptic norepinephrine transporter. Atomoxetine is sold in the form of the hydrochloride salt of atomoxetine under the trademark STRATTERA® which is prescribed as oral capsules having dosages of 10 mg, 18 mg, 25 mg, 40 mg, and 60 mg and as a generic under the trademark ATTENTIN® by Torrent Pharmaceuticals.

These xetine compositions of the prior art have however the drawback of showing a poor solubility in an acidic and lower acidic medium such as the gastric juice, where they usually show an incomplete dissolution. The bioavailability of the drug is thus reduced. In addition, xetine based drugs appeared to be extremely caustic, expensive, and have a bitter taste, which usually leads to constraining methods of treatment. There is thus a need to provide an improved composition which optimizes the intake of a single tablet, especially by increasing the bioavailability of the xetine drug.

Different improved compositions and dosage forms containing a xetine product have been developed. Patent application WO 2006/108120 relates to atomoxetine hydrochloride compositions which exhibit high stability properties, the stability criteria being the amount of impurities being formed during the time the product is stored. Patent applications US 2005/152974 and US 2006/057199 both relate to sustained release dosage forms, one comprising a matrix containing atomoxetine and a wax material, and the other one comprising a coated atomoxetine core having modified-release properties.

However, none of these improvements are dealing with the solubility of xetine compositions of the prior art in acidic and lower acidic mediums.

Various other patent applications such as WO 2000/08016 and US 2005/152974 disclose derivatives of the xetine drugs, wherein the parent compound is modified by making non toxic acid addition salts thereof. Thus, are disclosed salts prepared from organic acids such as fumaric, malic, or tartaric. However, none of these disclose the use of organic acids by themselves as an additive into the formulation of xetine compositions.

### SUMMARY OF THE INVENTION

The invention allows achieving high solubility in acidic and lower acidic medium such as of the stomach, which in turn allows plasmatic concentration sufficient to achieve an improved therapeutical effect.

This goal is achieved by providing a xetine composition comprising a xetine drug, and especially atomoxetine with a release enhancer organic acid.

The invention thus provides a xetine or salt thereof composition having a dissolution in a phosphate buffer at pH 6.8, using USP basket 10 Mesh at 75 rpm, of at least 15% at 60 minutes, of at least 40% at 120 minutes, of at least 70% at 300 minutes.

The invention also provides a xetine or salt thereof composition comprising at least one release enhancer organic acid.

According to another embodiment, the xetine or salt thereof is selected from paroxetine, tomoxetine, atomoxetine, reboxetine, dapoxetine, fluoxetine, sluoxetine, duloxetine, or combination thereof, preferably atomoxetine.

The invention also provides an atomoxetine or salt thereof composition having a dissolution in a phosphate buffer at pH 6.8, using USP basket 10 Mesh at 75 rpm, of at least 15% at 60 minutes, of at least 40% at 120 minutes, of at least 70% at 300 minutes.

According to one embodiment, the dissolution is of at least 17% at 60 minutes, of at least 50% at 120 minutes and of at least 90% at 300 minutes.

According to another embodiment, the composition is in the tablet form.

According to another embodiment, the release enhancer organic acid is selected from the group consisting of fumaric acid, tartaric acid, malic acid, citric acid, lactic acid, malonic acid, glutaric acid, ascorbic acid, propionic acid, succinic acid, glycolic acid, stearic acid, lactic acid, malic acid, pamoic acid, maleic acid, hydroxymaleic acid, phenylacetic acid, glutamic acid, benzoic acid, salicylic acid, 2-acetoxybenzoic acid, oxalic acid, HOOC—(CH2)n-COOH where n is 0-4, and mixture thereof.

Preferably, the release enhancer organic acid is malic acid or tartaric acid DL or a mixture thereof.

According to another embodiment, the xetine is in an amount of 5% to 90% wt.

According to another embodiment, the release enhancer organic acid is in an amount of 1 to 50% wt.

The invention further provides a method for the treatment of depression or ADHD conditions, comprising the step of orally administrating to a patient in need of such treatment a therapeutically effective unit dosage of a xetine or salt thereof composition having a dissolution phosphate buffer at pH 6.8 using USP basket 10 Mesh at 75 rpm, of at least 15% at 60 minutes, of at least 40% at 120 minutes, of at least 70% at 300 minutes.

The invention further provides a method for the treatment of depression or ADHD conditions, comprising the step of orally administrating to a patient in need of such treatment a therapeutically effective unit dosage of a xetine or salt thereof composition comprising at least one release enhancer organic acid.

The invention further provides a method for the manufacture of a xetine composition, comprising the steps of (i) preparing granules of the active ingredient together with a binder and the release enhancer organic acid, and (ii) compressing said granules with extra-granular excipients, into a tablet.

### BRIEF DESCRIPTION OF THE DRAWINGS

FIG. 1 is the dissolution profile of tablets I, II and III prepared according to example 1, in 1000 ml phosphate buffer pH 6.8 using basket 10 mesh at 75 rpm.

FIG. 2 is the dissolution profile of tablet IV prepared according to example 2, at different pH using basket 10 mesh at 75 rpm.

### DETAILED DESCRIPTION OF EMBODIMENTS OF THE INVENTION

The term “xetin” as used in the invention designates at least one norepidephrine reuptake inhibitor. The xetin is preferably selected from the group consisting of paroxetine, tomoxetine, atomoxetine, reboxetine, fluoxetine, poroxetine, sluoxetine and duloxetine, and salts thereof.

Preferred xetin are the commercially available tomoxetine and the atomoxetine and salts thereof.

The xetine compound of the invention can be used in the pharmaceutical compositions of the present invention either as the free amine or as any pharmaceutically acceptable salt thereof. The term “pharmaceutically acceptable salts” in the present invention includes derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like, and combinations comprising one or more of the foregoing salts. When considering atomoxetine, the hydrochloride salt of atomoxetine is preferred.

The amount of the active ingredient in the pharmaceutical compositions of the present invention will be a therapeutically effective amount. A therapeutically effective amount will generally be an amount within the range of from about 0.01 to about 90%, and preferably an amount within the range of from about 40 to about 70% by weight of the composition. It is understood that higher or lower weight percentages of the active ingredient may be present in the pharmaceutical compositions. By “therapeutically effective amount” as used herein is meant an amount of active component in the pharmaceutical compositions of the present invention which is effective to beneficially treat the patient in need thereof. An especially adapted range for the active ingredient content is 60 to 70% by weight.

The second component of the composition of the invention is the release enhancer organic acid. The term “release enhancer” as used in the invention designates the property to enhance the maximum solubility of a product as well as its dissolution rate. Such components organic acids are already known and available in the market. The inventor has found that surprisingly, when one or more release enhancer organic acids comes into the formulation of a xetine drug composition, its solubility in acidic and lower acidic medium is enhanced.

Said release enhancer organic acid is selected from any pharmaceutically acceptable organic acid, such as fumaric acid, tartaric acid, malic acid, citric acid, lactic acid, malonic acid, glutaric acid, ascorbic acid, propionic acid, succinic acid, glycolic acid, stearic acid, lactic acid, malic acid, pamoic acid, maleic acid, hydroxymaleic acid, phenylacetic acid, glutamic acid, benzoic acid, salicylic acid, 2-acetoxybenzoic acid, oxalic acid, HOOC—(CH2)n-COOH where n is 0-4, and the like or mixture thereof. Preferred organic acids are organic diacids. Preferred acids contain up to 6 carbon atoms. Most preferably the acid used in the invention is tartaric acid, malic acid, fumaric acid or mixtures thereof.

Malic, aspartic, tartaric and gluconic acids exists in enantiomeric forms and this invention includes salts with both the D and L-acids and racemic mixtures thereof. Most preferred release enhancer organic acids are tartaric acid DL and malic acid, as well as mixtures thereof.

The pharmaceutical compositions of the present invention will generally contain the organic acid release enhancer between about 1 to about 50% by weight percent; preferably between about 1 to about 30 wt %; and even more preferably between 10 and 20 wt % by weight of the pharmaceutical composition. A particularly preferred percentage of the organic acid release enhancer is about 14%.

The invention thus provides a pharmaceutical composition having an enhanced solubility in acidic and lower acidic medium. The term “acidic and lower acidic medium” in this invention designates a pH of a medium which is in the range of 3 to 7. Preferably, the composition exhibits dissolution in phosphate buffer at pH 6.8, using the US Pharmacopeia type I method, basket 10 Mesh at 75 rpm, of at least 15% at 60 minutes, of at least 40% at 120 minutes, of at least 70% at 300 minutes, and preferably of at least 17% at 60 minutes, of at least 50% at 120 minutes and of at least 90% at 300 minutes.

Such values could not be reached with compositions of the prior art. The invention thus allows reaching high bioavailabilities, which could not be obtained with the prior art formulations.

The preferred dosage forms of the pharmaceutical compositions of the present invention are solid dosage forms adapted for oral administration. Tablet dosage forms are the particularly preferred solid dosage forms of the stabilized pharmaceutical compositions of the present invention. Tablet dosage forms may contain for example, as excipients, any pharmaceutically acceptable lubricant, binder, disintegrant, diluent, carrier, preservative or combination thereof. Solid dosage forms that are not formulated as tablets typically do not need a lubricant component since this is typically added to facilitate manufacture of tablet dosage forms. For the purpose of oral preparations of the present invention, pharmaceutically acceptable inert carriers or diluent or filler can be either solid or not. Among other preferred dosage forms useful for formulating the stabilized pharmaceutical compositions of the present invention include powders, dispersible granules, capsules and cachets.

The pharmaceutical compositions of the present invention may also contain any pharmaceutically acceptable excipient or combination thereof. Conventional pharmaceutical excipients include those which function in a dosage form, for example, as a lubricant, glidant, diluent, binder, plasticizer, disintegrant, carrier, colorant, preservative or coating material.

Examples of pharmaceutically acceptable excipients include, but are not limited to, lactose, dextrates, dextrin, dextrose, mannitol, dicalcium phosphate, xylitol, sugar, saccharose, corn starch, hydrolyzed starch (malto-dextrine), modified corn starch, maize starch, dried starch sodium starch glycolate, mannitol, sorbitol, silicon dioxide, colloidal silicone dioxide, dicarboxylic or tricarboxylic ester-based plasticizers, benzoates, epoxidized vegetable oils, sulfonamides, organophosphates, glycols or polyethers, polyethylene glycol, dibutyl sebacate, acetylated monoglycerides, alkyl citrates, povidone or copovidone, microcrystalline cellulose, croscarmellose sodium, polyvinylpyrrolidone, polyvinylalcohol, hydroxypropylcellulose, hydroxymethylcellulose, hydroxypropylmethylcellulose, alkylcellulose such as methylcellulose or ethylcellulose, gelatin, cross-linked polyvinylpyrrolidone (PVP) e.g products known under the trademark Avicel®, Filtrak®, Heweten® or Pharmacel®, sodium carboxymethyl starch, magnesium stearate, sodium stearyl fumarate, polyethylene glycol, stearic acid, hydrogenated vegetable oil, glyceryl behenate and talc, colloidal silica e.g known under the trademark Aerosil®, tribasic calcium phosphate, and mixtures thereof.

Further excipients are disclosed in “Handbook of Pharmaceutical excipients”, 2^{nd} Ed., 1994, American Pharmaceutical Association, Washington, ISBN 0 91730 66 8, by Wade A., Weller P J.).

The pharmaceutical compositions according to the invention also cover dosage forms such as controlled release, sustained release, pulsed release, or enteric coated forms. Controlled release formulations allow many inherent therapeutic benefits that are not achieved with corresponding short acting immediate release preparations. Indeed, for attention deficit disorders, patients must maintain their blood levels with constant amount of drug at a therapeutically effective level to provide symptomatic relief which is not achieved with conventional immediate release formulations. Sustained-release formulations can be based on matrix technology and allow the drug to be administered only once daily or even less frequently. Enteric coatings might be desirable to protect the stomach against the irritant effects of the xetine drug. Such technologies are available to the man skilled in the art.

Another goal is to provide methods of preparing the improved composition according to the invention. The pharmaceutical compositions may be manufactured by various techniques available to the skilled man.

One preferred process comprises the steps of preparing granules of the active ingredient together with a binder and the release enhancer organic acid, and then compressing said granules with extra-granular excipients, into a tablet.

Granules manufacture can take place according to various techniques, such fluidized bed technology, tank mixing, or crushing compacts previously manufactured. Granules are optionally, but not necessarily, compressed into tablets. One embodiment includes the steps of dry mixing the components, compressing into a compressed form and crushing said compressed form into granules.

When the granule obtained (whether subsequently coated or not) is compressed to form tablets, this step can be implemented using any conventional technique which is suitable, for example using alternating or rotating compressing machines.

Direct compression is also available for the manufacture of the instant pharmaceutical compositions.

Coating techniques are also available for the manufacture of a final coated pharmaceutical composition.

The invention also provides methods for the treatment of attention-deficit hyperactivity disorder (ADHD) depressive diseases and diseases usually treated with the mentioned xetine drugs, comprising the step of orally administering to a patient in need of such treatment a therapeutically effective unit dosage form of the composition according to the invention.

### EXAMPLES

The following example illustrates the invention without limiting it.

### Example 1

The following controlled release formulations were prepared by granulating with the povidone solution containing the acid. The obtained granules are blended with sodium stearyl fumarate and colloidal silicon dioxide, and compressed into tablets. The obtained tablets are then coated with the ethylcellulose-povidone solution.

| | | | Amount (mg) | | | |
|---|---|---|---|---|---|---|
| | Excipients | Fonction | Tablet I | Tablet II | Tablet III | Tablet IV |
| Tablet core | Atomoxetine | API | 91.07 | 91.07 | 91.07 | 91.07 |
| | HCl | | | | | |
| | Tartaric Acid DL | Release | — | 20 | — | 20 |
| | | enhancer | | | | |
| | MalicAcid | Release | — | — | 20 | — |
| | | enhancer | | | | |
| | Povidone K30 or | Binder | 15 | 18 | 15 | 18 |
| | copovidone K28 | | | | | |
| | Sodium Stearyl | Lubricant | 1.59 | 1.95 | 1.59 | 1.95 |
| | Fumarate | | | | | |
| | Colloidal silicon | Glidant | 0.7 | 0.65 | 0.7 | 0.65 |
| | Dioxide | | | | | |
| Tablet coating | Ethylcellulose | Insoluble | 5 | 5 | 5 | 5.58 |
| | | polymer | | | | |
| | Povidone K30 or | Coating | 3.33 | 3.33 | 3.33 | 2.75 |
| | copovidone K28 | porosity | | | | |
| | Polyethylene | Plasticizer | 0.42 | 0.42 | 0.42 | 0.42 |
| | Glycol | | | | | |
| | Dibutyl | Plasticizer | 1.25 | 1.25 | 1.25 | 1.25 |
| | Sebacate | | | | | |
| | | Total | 118.36 | 141.67 | 138.36 | 141.67 |

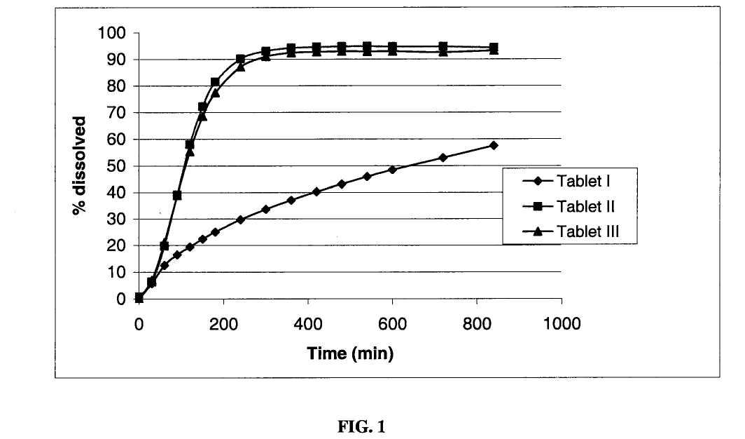

Dissolution tests were run on the tablets I, II, and III, in 1000 ml phosphate buffer pH 6.8 using basket 10 mesh at 75 rpm. The results presented in FIG. 1 clearly show that the dissolution is very slow when there is no acid in the tablet core. Malic or tartaric acids help to get a faster release at pH 6.8.

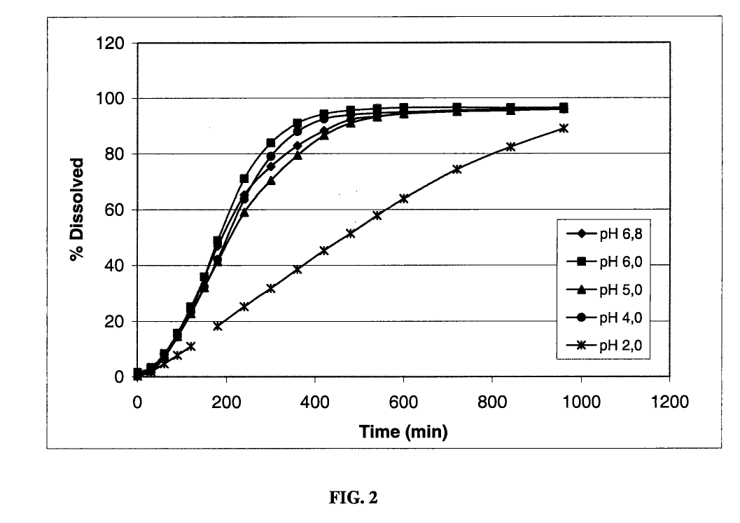

Dissolution tests were run on the tablet IV at different pH, using basket 10 mesh at 75 rpm. The results that are presented in FIG. 2, show that, at different acidic and lower acidic pH, the composition according to the invention of tablet IV reached a maximum dissolution percentage which is higher than what was accessible with the prior art composition at less constraining conditions (pH 6.8, FIG. 1).

The dissolution profile are given for a composition comprising 80 mg equivalent xetin, resp. atomoxetine, i.e. a composition comprising 80 mg of equivalent xetin, resp. atomoxetine.

1. A xetine or salt thereof composition having a dissolution in a phosphate buffer at pH 6.8, using USP basket 10 Mesh at 75 rpm, of at least 15% at 60 minutes, of at least 40% at 120 minutes, of at least 70% at 300 minutes.

2. The composition of claim 1, wherein the dissolution is of at least 17% at 60 minutes, of at least 50% at 120 minutes and of at least 90% at 300 minutes.

3. The composition of claim 1, wherein the xetine or salt thereof is selected from paroxetine, tomoxetine, atomoxetine, reboxetine, dapoxetine, fluoxetine, sluoxetine, duloxetine, or combination thereof.

4. The composition of claim 1, wherein the xetine is in an amount of 5% to 90% wt.

5. The composition of claim 1, in a tablet form.

6. The composition of claim 1, wherein the xetine is atomoxetine.

7. The composition of claim 6, wherein the dissolution is of at least 17% at 60 minutes, of at least 50% at 120 minutes and of at least 90% at 300 minutes.

8. The composition of claim 7, in a tablet form.

9. A xetine or salt thereof composition comprising at least one release enhancer organic acid.

10. The composition of claim 9, wherein the xetine or salt thereof is selected from paroxetine, tomoxetine, atomoxetine, reboxetine, dapoxetine, fluoxetine, sluoxetine, duloxetine, or combination thereof.

11. The composition of claim 9, wherein the release enhancer organic acid is selected from the group consisting of fumaric acid, tartaric acid, malic acid, citric acid, lactic acid, malonic acid, glutaric acid, ascorbic acid, propionic acid, succinic acid, glycolic acid, stearic acid, lactic acid, malic acid, pamoic acid, maleic acid, hydroxymaleic acid, phenylacetic acid, glutamic acid, benzoic acid, salicylic acid, 2-acetoxybenzoic acid, oxalic acid, HOOC—(CH2)n-COOH where n is 0-4, and mixture thereof.

12. The composition of claim 9, wherein the release enhancer organic acid is malic acid or tartaric acid DL or a mixture thereof.

13. The composition of claim 9, wherein the xetine is in an amount of 5% to 90% wt.

14. The composition of claim 9, wherein the release enhancer organic acid is in an amount of 1 to 50% wt.

15. The composition of claim 9, in a tablet form.

16. The composition of claim 9, wherein the xetine or salt thereof is atomoxetine.

17. The composition of claim 12, wherein the xetine or salt thereof is atomoxetine.

18. The composition of claim 16, in a tablet form.

19. The composition of claim 17, in a tablet form.

20. The composition of claim 9, wherein the xetine or salt thereof is atomoxetine, wherein the release enhancer organic acid is selected from the group consisting of fumaric acid, tartaric acid, malic acid, citric acid, lactic acid, malonic acid, glutaric acid, ascorbic acid, propionic acid, succinic acid, glycolic acid, stearic acid, lactic acid, malic acid, pamoic acid, maleic acid, hydroxymaleic acid, phenylacetic acid, glutamic acid, benzoic acid, salicylic acid, 2-acetoxybenzoic acid, oxalic acid, HOOC—(CH2)n-COOH where n is 0-4, and mixture thereof, wherein the release enhancer organic acid is in an amount of 1 to 50% wt, and which is in a tablet form.

21. A method for the treatment of depression or ADHD conditions, comprising the step of orally administrating to a patient in need of such treatment a therapeutically effective unit dosage of a xetine or salt thereof composition having a dissolution phosphate buffer at pH 6.8 using USP basket 10 Mesh at 75 rpm, of at least 15% at 60 minutes, of at least 40% at 120 minutes, of at least 70% at 300 minutes.

22. A method for the treatment of depression or ADHD conditions, comprising the step of orally administrating to a patient in need of such treatment a therapeutically effective unit dosage of a xetine or salt thereof composition comprising at least one release enhancer organic acid.

23. A method for the manufacture of a xetine composition, comprising the steps of (i) preparing granules of the active ingredient together with a binder and the release enhancer organic acid, and (ii) compressing said granules with extra-granular excipients, into a tablet.
